(19) United States
(12) Patent Application Publication
(10) Pub. No.: US 2019/0368150 A1
(43) Pub. Date: Dec. 5, 2019

(54) RISK DETERMINATION DEVICE, RISK DETERMINATION SYSTEM, RISK DETERMINATION METHOD, AND COMPUTER-READABLE RECORDING MEDIUM

(71) Applicant: NEC CORPORATION, Tokyo (JP)

(72) Inventor: Shinji KASAHARA, Tokyo (JP)

(73) Assignee: NEC CORPORATION, Tokyo (JP)

(21) Appl. No.: 16/477,579

(22) PCT Filed: Dec. 27, 2017
(86) PCT No.: PCT/JP2017/046824

§ 371 Date: Jul. 12, 2019

(30) Foreign Application Priority Data
Jan. 13, 2017 (JP) 2017-004595

(51) Int. Cl. E02D 17/20; G01N 33/24; A01G 25/16

(52) U.S. Cl. CPC E02D 17/20; A01G 25/167; G01N 33/246

(57) ABSTRACT

In order to evaluate the risk of a slope collapsing before installing a sensor, a risk determination device 100 comprises: a first calculation unit 110 that calculates a parameter to indicate the condition of soil on the basis of the relationship between the condition of the soil constituting a slope and the moisture condition of the soil and virtual data for the moisture condition; a second calculation unit 120 that calculates a safety factor for the slope using the calculated parameter; and a determination unit 130 that determines the risk of the slope collapsing on the basis of the moisture condition in which the calculated safety factor is below a threshold and the moisture condition of the soil based on the virtual data when the moisture is saturated.

### TECHNICAL FIELD

The present disclosure relates to a risk determination device and the like.

### BACKGROUND ART

A safety factor obtained by a slope stability analytical expression is generally used as an index for evaluating safety of a slope. As a technology relating to evaluation of safety of a slope, PTL 1 discloses an invention in which a safety factor of a slope is calculated based on data that are output from a sensor installed on the slope.

### CITATION LIST

### Patent Literature

[PTL 1] International Publication No. WO 2016/027390

### SUMMARY OF INVENTION

### Technical Problem

A plurality of slopes to be monitored, i.e., a plurality of slopes having a risk of slope collapsing exist. However, in a case where the number of sensors that can be installed is limited, there is a possibility that sensors cannot be installed on all the slopes having a collapsing risk. In the technology in PTL 1 or the like, safety of a slope can be evaluated, but it is not until the sensor is installed that the evaluation can be performed. In other words, the technology in PTL 1 or the like has a problem that it is difficult to evaluate a risk of slope collapsing before installing a sensor.

An exemplary object of the present disclosure is to solve the above-mentioned problem that it is difficult to evaluate a risk of slope collapsing before installing a sensor.

### Solution to Problem

According to one aspect, provided is a risk determination device including: a first calculation means for calculating a parameter indicating a condition of soil constituting a certain slope, based on a relationship between the condition of the soil and a moisture condition of the soil and virtual data of the moisture condition; a second calculation means for calculating a safety factor of the slope by use of the parameter being calculated; and a determination means for determining a collapsing risk of the slope, based on the moisture condition in which the safety factor being calculated is less than a threshold value, and the moisture condition of the soil based on the virtual data when the moisture is saturated.

According to another aspect, provided is a risk determination system including: a risk determination device which includes a first calculation means for calculating a parameter indicating a condition of soil constituting a certain slope, based on a relationship between the condition of the soil and a moisture condition of the soil and virtual data of the moisture condition, a second calculation means for calculating a safety factor of the slope by use of the parameter being calculated, and a determination means for determining a collapsing risk of the slope, based on the moisture condition in which the safety factor being calculated is less than a threshold value, and the moisture condition of the soil based on the virtual data when the moisture is saturated; and a setting device for setting virtual data.

According to yet another aspect, provided is a risk determination method including: calculating a parameter indicating a condition of soil constituting a certain slope, based on a relationship between the condition of the soil and a moisture condition of the soil and virtual data of the moisture condition; calculating a safety factor of the slope by use of the parameter being calculated; and determining a collapsing risk of the slope, based on the moisture condition in which the safety factor being calculated is less than a threshold value, and the moisture condition of the soil based on the virtual data when the moisture is saturated.

According to yet another aspect, provided is a computer-readable recording medium which non-temporarily stores a program causing a computer to execute: a step of calculating a parameter indicating a condition of soil constituting a certain slope, based on a relationship between the condition of the soil and a moisture condition of the soil and virtual data of the moisture condition; a step of calculating a safety factor of the slope by use of the parameter being calculated; and a step of determining a collapsing risk of the slope, based on the moisture condition in which the safety factor being calculated is less than a threshold value, and the moisture condition of the soil based on the virtual data when the moisture is saturated.

### Advantageous Effects of Invention

According to the present disclosure, a risk of slope collapsing can be evaluated before installing a sensor.

### BRIEF DESCRIPTION OF DRAWINGS

FIG. 1 is a block diagram illustrating an example of a configuration of a risk determination device.

FIG. 2 is a schematic diagram exemplifying a relationship between a safety factor and a moisture amount in soil of a slope.

FIG. 3 is a flowchart illustrating an example of processing executed by the risk determination device.

FIG. 4 is a block diagram illustrating another example of a configuration of the risk determination device.

FIG. 5 is a flowchart illustrating another example of processing executed by the risk determination device.

FIG. 6 is a block diagram illustrating an example of a configuration of a risk determination system.

FIG. 7 is a flowchart illustrating an example of processing executed by a setting device.

FIG. 8 is a table showing an example of relational expressions between soil parameters and moisture amounts in soil and moisture amounts in soil at the time of saturation for a plurality of slopes.

FIG. 9 is a table showing an example of topographic data for the plurality of slopes.

FIG. 10 is a graph illustrating an example of relationships between safety factors and moisture amounts in soil which are calculated for the plurality of slopes.

FIG. 11 is a block diagram illustrating an example of a hardware configuration of a computer device.

### EXAMPLE EMBODIMENT

### First Example Embodiment

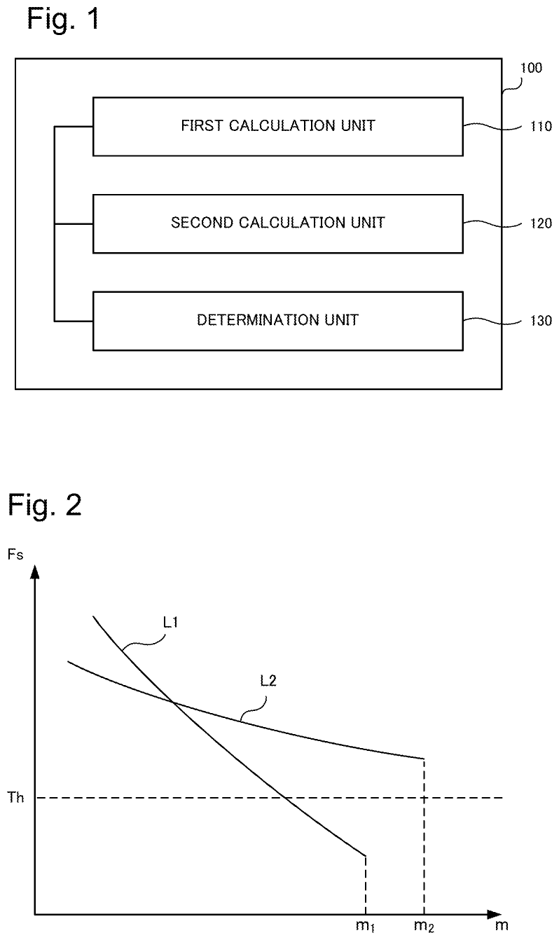

FIG. 1 is a block diagram illustrating a configuration of a risk determination device 100 according to an example embodiment of the present disclosure. The risk determination device 100 is an information processing device for evaluating a risk of slope collapsing.

The slope described herein is a part of a ground surface, and more particularly, a ground point having a possibility of slope collapsing such as a landslide. A liability of slope collapsing depends not only on an angle of a slope but also on various causes of soil and the like constituting the slope. Therefore, an upper limit and a lower limit of an angle of the slope described herein cannot be defined within a constant range.

Further, the risk of slope collapsing refers to a risk in that a slope collapses. The collapsing risk described herein may be an alternative between “a high possibility (of slope collapsing)” and “a low possibility (of slope collapsing)”, but may be expressed in more multiple stages. Further, a method of expressing the collapsing risk may be a numerical value, a character, a symbol, a color, a sound, and the like, and is not particularly limited.

The risk determination device 100 includes a first calculation unit 110, a second calculation unit 120, and a determination unit 130. Further, the risk determination device 100 may include other configurations as needed. For example, the risk determination device 100 may include a configuration of outputting a collapsing risk determined by the determination unit 130 (such as a display or a speaker.

The first calculation unit 110 calculates parameters indicating a state of soil constituting a slope (herein also referred to “soil parameters”). The soil parameters described herein can also be said as parameters relating to a liability of slope collapsing. Specifically, the soil parameters include a soil clod weight, a pore water pressure, viscosity, an internal friction coefficient, and the like of the soil. The first calculation unit 110 calculates at least any of those parameters.

The first calculation unit 110 calculates the soil parameters based on a relationship between a condition of soil constituting the slope and a moisture condition of the soil. In some cases, the first calculation unit 110 calculates the soil parameters based on an expression indicating the relationship between the soil condition and the moisture condition. The expression may be a known expression, but may be calculated by the first calculation unit 110.

Further, the first calculation unit 110 calculates the soil parameters based on virtual data of the moisture condition of the soil. More specifically, the first calculation unit 110 calculates the soil parameters based on the relationship between the condition of the soil constituting the slope and the moisture condition of the soil and the virtual data of the moisture condition of the soil. For example, the first calculation unit 110 calculates the soil parameters corresponding to values of the virtual data by substituting the virtual data in the relational expressions indicating a mutual relationship between the parameters indicating the soil state and the moisture state of the soil.

The virtual data contain virtual or schematic numerical values of the parameters indicating the moisture condition of the soil, and for example, values obtained by a test (test values) or values described in a literature (literature values). For example, the parameters indicating the moisture condition of the soil include a moisture amount and a saturation degree of the soil. The saturation degree described herein is a ratio of a water volume in pores to a pore volume in the soil. Further, the moisture amount described herein may be any of a volume water content (a ratio of a water volume to a soil volume) and a weight water content (a ratio of a water weight to a soil weight). In other words, the parameters indicating the moisture condition of the soil can also be said as parameters indicating an extent to which the soil contains the moisture.

The second calculation unit 120 calculates a safety factor of the slope. More specifically, the second calculation unit 120 calculates a safety factor based on a predetermined stability analytical expression (slope stability analytical expression) in a slope stability analysis. As the slope stability analytical expression, stability analytical expressions of the Fellenius method, the modified Fellenius method, the Bishop method, the Janbu method, and the like are generally known. Further, various slope stability analytical expressions obtained by applying or modifying those stability analytical expressions are also known. The second calculation unit 120 can calculate a safety factor by use of any of those slope stability analytical expressions. In other words, the slope stability analytical expression adopted to the calculation of a safety factor are not necessarily limited to a specific expression.

To put it simply, the safety factor of the slope is a ratio of a sliding force on the slope (a force exerted to slide) and a resisting force against the sliding force. In general, the stability of the slope is higher as a value of the safety factor is higher. Specifically, safety is confirmed when the value is 1 or greater. The safety factor can be said as one example of an index indicating the stability of the slope.

The second calculation unit 120 calculates the stability factor by use of the soil parameters calculated by the first calculation unit 110. For example, the second calculation unit 120 calculates the safety factor by substituting the soil parameters, which are calculated by the first calculation unit 110, in the predetermined stability analytical expression. The soil parameters calculated by the first calculation unit 110 are the parameters calculated based on the virtual data, and hence do not necessarily match with the actual soil parameters of the slope. Therefore, the safety factor calculated by the second calculation unit 120 can be said as a virtual value.

The determination unit 130 determines the risk of slope collapsing. More specifically, the determination unit 130 determines the collapsing risk based on the safety factor calculated by the second calculation unit 120 and the moisture condition of the soil based on the virtual data when the moisture is saturated. Specifically, the determination unit 130 is capable of determining the risk of slope collapsing by comparing a moisture condition in which the safety factor calculated by the second calculation unit 120 is a threshold value or less and the moisture condition of the soil based on the virtual data when the moisture is saturated.

FIG. 2 is a schematic diagram exemplifying a relationship between a safety factor and a moisture amount in soil of a slope. In this example, curve lines L1 and L2 indicate safety factors (Fs) corresponding to moisture amounts in soil (m) on different slopes. In general, the safety factor is decreased as the moisture amount in soil is increased. The moisture amount in soil at the time of saturation for the curve line L1 is indicated with “m1”, and the moisture amount in soil at the time of saturation for the curve line L2 is indicated with “m2”.

In this example, in the case of the slope having the safety factor indicated by the curve line L1, the safety factor is less than a threshold value Th (for example, 1.0) before the moisture amount in soil is saturated. In comparison, in the case of the slope having the safety factor indicated by the curve line L2, the safety factor is equal to or greater than the threshold value Th even when the moisture amount in soil is saturated. Therefore, it can be said that the slope having the safety factor indicated by the curve line L2 has a lower collapsing risk than the slope having the safety factor indicated by the curve line L1. This is because the safety factor is not less than a threshold value Th even when the slope having the safety factor indicated by the curve line L2 retains the moisture until the saturation.

As in this example, the determination unit 130 determines the collapsing risk based on a moisture condition in which a safety factor of a certain slope is less than a specific threshold value and a moisture condition in which the moisture of the slope is saturated (based on the virtual data). For example, the determination unit 130 determines that the slope having the safety factor indicated by the curve line L1 has a high collapsing risk (i.e., more risky) and that the slope having the safety factor indicated by the curve line L2 has a low collapsing risk (i.e., safer).

Alternatively, the determination unit 130 may determine the risk of slope collapsing in a more stepwise manner by using a plurality of threshold values. For example, the determination unit 130 may determine the risk of slope collapsing in four steps including “0 (safe)”, “1 (relatively risky)”, and “3 (very risky)” by using three threshold values.

A configuration of the risk determination device 100 is as described above. With such a configuration, the risk determination device 100 determines a collapsing risk of a slope provided by virtual data. For example, a user prepares virtual data on one slope or a plurality of slopes, which are desired to be subjected to the collapsing risk determination, through tests or the like conducted in advance. The virtual data required in this case are, for example, parameters (a moisture amount or a saturation degree) from a state containing moisture in a soil to a saturated state or parameters from the state containing moisture in the soil to a state in which the safety factor is less than 1.

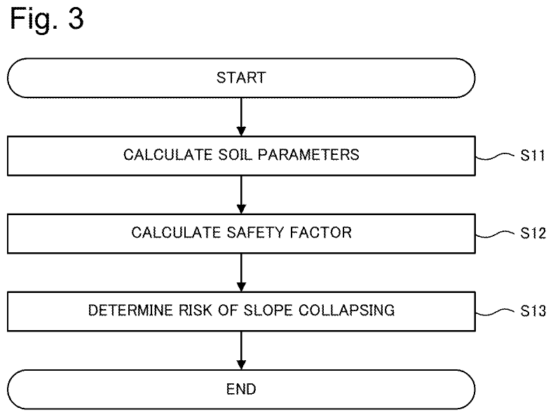

FIG. 3 is a flowchart illustrating processing executed by the risk determination device 100. In Step S11, the first calculation unit 110 calculates the soil parameters of the slope being a determination target (i.e., the slope being subjected to the collapsing risk determination). The first calculation unit 110 calculates the soil parameters required for calculating the safety factor by acquiring the virtual data from the outside or reading out the virtual data from a storage device.

In Step S12, the second calculation unit 120 calculates the safety factor of the slope being a determination target by use of the soil parameters calculated in Step S11. The second calculation unit 120 calculates the safety factor of the slope in various moisture conditions by use of a predetermined slope stability analytic expression. In other words, it can be said that the second calculation unit 120 calculates a transition of the safety factor in accordance with the change in moisture condition.

In Step S13, the determination unit 130 determines the collapsing risk of the slope being a determination target, based on the safety factor calculated in Step S12. The determination unit 130 determines the risk of slope collapsing based on the moisture condition in which the safety factor is less than a predetermined threshold value and the moisture condition in which the moisture of the slope being a determination target is saturated.

As described above, the risk determination device 100 according to the present example embodiment is capable of determining the risk of slope collapsing based on the virtual data. Therefore, the risk determination device 100 enables the determination of the risk of slope collapsing without using measured values of the moisture condition of the slope (i.e., data measured at an actual field). Therefore, according to the risk determination device 100, the risk of slope collapsing can be evaluated before installing a sensor.

The collapsing risk evaluated by the risk determination device 100 can be used for determination of an order of priority by which sensors are installed on slopes. In other words, a user can install a sensor preferentially from a slope having a high collapsing risk determined by the risk determination device 100. In other words, it can be said that the risk determination device 100 can provide a user with an objective evaluation criterion at the time of installing sensors on slopes.

### Second Example Embodiment

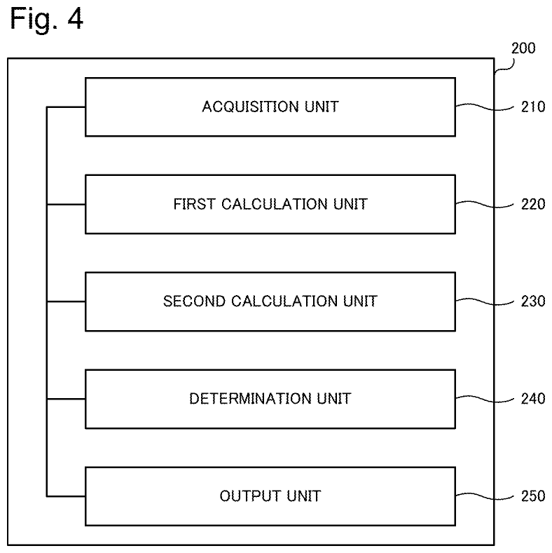

FIG. 4 is a block diagram illustrating a configuration of a risk determination device 200 according to another example embodiment.

The risk determination device 200 includes an acquisition unit 210, a first calculation unit 220, a second calculation unit 230, a determination unit 240, and an output unit 250.

In the risk determination device 200, the first calculation unit 220, the second calculation unit 230, and the determination unit 240 have configurations with the same names and the similar functions as those in the first example embodiment. In the present example embodiment, those configurations are described mainly focusing on differences with the first calculation unit 110, the second calculation unit 120, and the determination unit 130 in the first example embodiment.

The acquisition unit 210 acquires data to be used for determination of a risk of slope collapsing. The acquisition unit 210 may acquire data from a storage medium of the risk determination device 200, or may acquire data from other devices through wires or wirelessly. The acquisition unit 210 acquires, for example, virtual data. Further, the acquisition unit 210 may acquire topographic data indicating topography of a slope being a determination target or vegetation data indicating vegetation of the slope being a determination target. The topographic data described herein contain numerical values indicating a slope length, a depth of a sliding layer from a ground surface, a slope angle, and the like. Further, the vegetation data described herein contain numerical values indicating presence or absence, kinds, density and the like of vegetation on the slope.

The first calculation unit 220 is similar to the first calculation unit 110 in the first example embodiment in that soil parameters are calculated. In addition, the first calculation unit 220 may specify a relational expression indicating a relationship between a condition of soil and the moisture condition thereof through calculation, based on the data acquired by the acquisition unit 210. In the present example embodiment, the first calculation unit 220 is configured to calculate soil parameters by use of the relational expression.

The second calculation unit 230 is similar to the second calculation unit 120 in the first example embodiment in that a safety factor is calculated. In addition to the virtual data acquired by the acquisition unit 210, the second calculation unit 230 may calculate the safety factor by use of at least any one of the topographic data and the vegetation data.

In general, a moisture amount in soil without vegetation is more likely to be increased and decreased as compared to that of soil with vegetation. Further, a tendency of changing the moisture amount in soil differs depending on kinds of vegetation. Similarly, the tendency of changing the moisture amount in soil also differs depending on specific topography of the slope. Therefore, the second calculation unit 230 can improve an accuracy of the safety factor by calculating the safety factor by use of the topographic data or the vegetation data as compared to the case without using the topographic data or the vegetation data.

The determination unit 240 is similar to the determination unit 130 in the first example embodiment in that the risk of slope collapsing is determined. In addition, the determination unit 240 supplies data indicating a collapsing risk to the output unit 250.

The output unit 250 outputs the data indicating the collapsing risk. For example, the output unit 250 may include a display device that displays the collapsing risk in a visible manner and a communication interface that transmits the data indicating the collapsing risk to other devices. Note that the display by the output unit 250 may include display of the collapsing risk with numbers or characters and display of the collapsing risk with colors on a map.

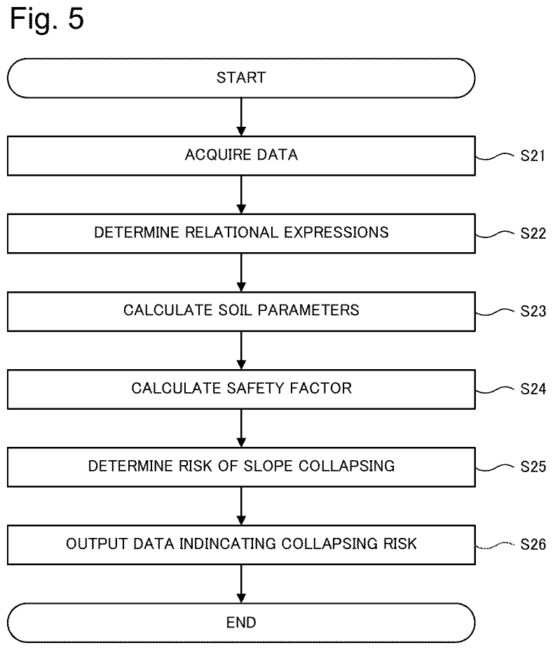

FIG. 5 is a flowchart illustrating processing executed by the risk determination device 200. In Step S21, the acquisition unit 210 acquires data required for the determination of the risk of slope collapsing. In Step S22, the first calculation unit 220 specifies a relational expression indicating the relationship between the condition of soil and the moisture condition thereof. Specifically, the first calculation unit 220 specifies the relational expression by reading out the relational expressions, which are stored in advance, in accordance with slopes. In Step S23, the first calculation unit 220 calculates the soil parameters by use of the relational expression specified in Step S22 and the virtual data acquired in Step S21.

In Step S24, the second calculation unit 230 calculates the safety factor by using the soil parameters calculated in Step S23. In Step S25, the determination unit 240 determines the risk of slope collapsing, based on the safety factor calculated in Step S24. In Step S26, the output unit 250 outputs (for example, displays) the data indicating the collapsing risk determined in Step S25.

As described above, the risk determination device 200 according to the present example embodiment can exert the actions and effects similar to those in the first example embodiment. Further, the risk determination device 200 can improve an accuracy of the safety factor by calculating the safety factor by use of the topographic data or the vegetation data.

### Third Example Embodiment

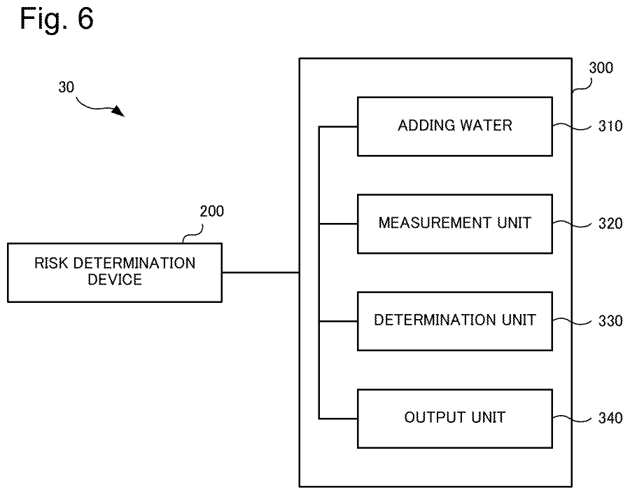

FIG. 6 is a block diagram illustrating a configuration of a risk determination system 30 according to further another example embodiment. The risk determination system 30 includes a setting device 300 in addition to the risk determination device 200 according to the second example embodiment.

The setting device 300 is an information processing device that sets data (virtual data and the like) used by the risk determination device 200. The data setting described herein refers to supply of the data to the risk determination device 200 in such a way that the risk determination device 200 can use the data. In the present example embodiment, the setting device 300 conducts a predetermined test (hereinafter also referred to as an “adding water test”) to a soil sample. The setting device 300 is connected to, for example, the risk determination device 200 through wires or wirelessly. Alternatively, the setting device 300 may be a part of the risk determination device 200. The setting device 300 includes an adding water unit 310, a measurement unit 320, a determination unit 330, and an output unit 340.

The adding water unit 310 adds moisture in a soil tank filled with a soil sample. For example, the adding water unit 310 is configured to pour moisture in the soil tank by a certain amount. The adding water unit 310 pours the moisture until the moisture of the soil in the soil tank is in a saturated state. The soil being a sample is collected by a small amount from an actual field (i.e., a slope being a determination target).

The measurement unit 320 measures a parameter indicating a moisture condition of the soil. In the present example embodiment, it is assumed that the parameter indicating the moisture condition of the soil is a moisture amount in soil (m). For example, the measurement unit 320 measures the moisture amount in soil by use of a sensor (a soil moisture meter or the like) installed in the soil tank.

Further, the measurement unit 320 may also measure parameters indicating the soil condition together. In the present example embodiment, it is assumed that the parameters indicating the soil condition include a soil clod weight (W), a pore water pressure (u), viscosity (c), and an internal friction coefficient (φ). In other words, the measurement unit 320 may further include sensors for measuring those parameters.

The determination unit 330 determines whether the moisture of the soil sample in the soil tank is saturated. For example, the determination unit 330 may determine the saturation of the soil based on a ground water level in the soil tank, or based on the moisture condition of the soil surface in the soil tank.

The output unit 340 outputs the parameters measured by the measurement unit 320. The output unit 340 outputs the moisture amount in soil at the time of saturation, which is determined by the determination unit 330. The output unit 340 may output not only the moisture amount in soil at the time of saturation but also other parameters. The parameters output by the output unit 340 are supplied to the risk determination device 200. Note that the parameters output by the output unit 340 may be recorded in a portable memory medium, and may be supplied to the risk determination device 200 via the memory medium.

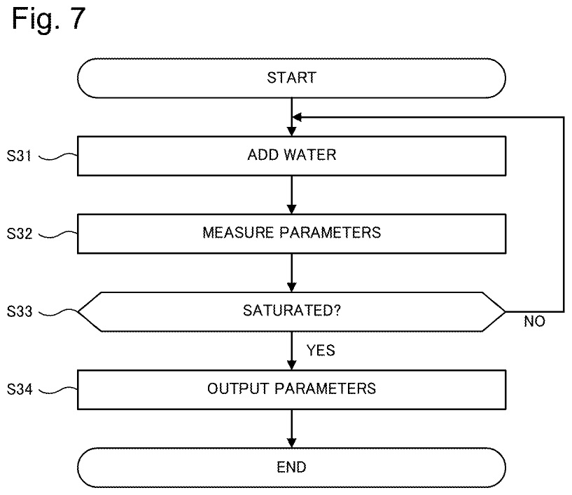

FIG. 7 is a flowchart illustrating processing executed by the setting device 300. In Step S31, the adding water unit 310 adds a predetermined amount of moisture in a soil sample. In Step S32, the measurement unit 320 measures various parameters in this moisture condition.

In Step S33, the determination unit 330 determines whether or not the moisture of the soil sample is saturated. When the moisture of the soil sample is in the saturated state (YES in Step S33), the output unit 340 outputs the parameters in Step S34. On the other hand, when the moisture of the soil sample is not in the saturated state (NO in Step S33), the adding water unit 310 repeats Step S31 again. The measurement unit 320 measures parameters in each moisture condition until the moisture of the soil sample is saturated.

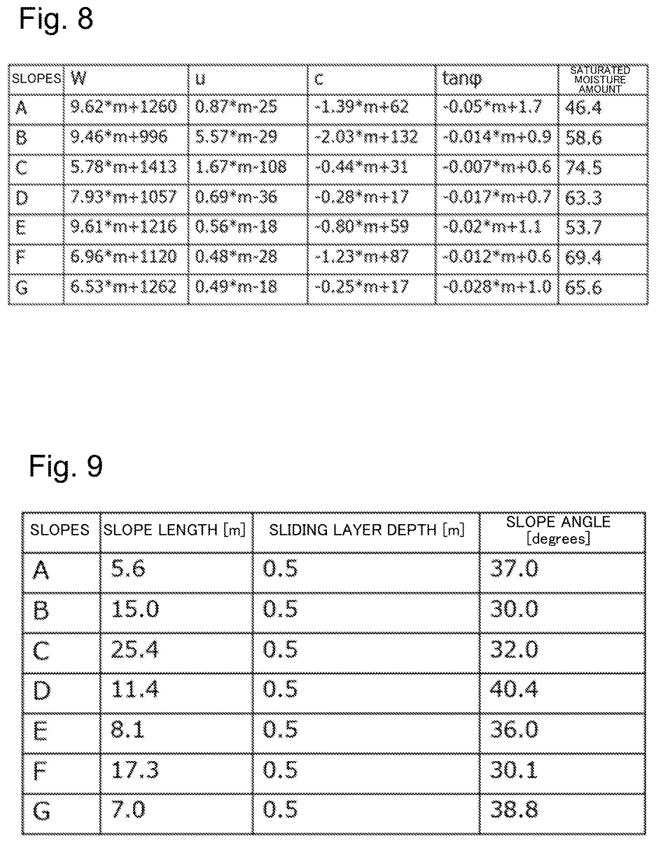

FIG. 8 is a table exemplifying relational expressions between various soil parameters and moisture amounts in soil and moisture amounts in soil at the time of saturation for a plurality of slopes (A to G). In this example, the soil clod weight (W) of the slope A is indicated with “9.62m+1260” by use of the moisture amount in soil (m). Further, the pore water pressure (u) of the slope A is indicated with “0.87m-25” by use of the moisture amount in soil (m). Note that the relational expressions between the soil parameters and the moisture amount in soil may not be linear functions of the moisture amount in soil.

The risk determination device 200 acquires at least the moisture amount in soil of each of the slopes among the parameters in FIG. 8 from the setting device 300. Further, the risk determination device 200 may acquire the soil parameters of each of the slopes or the relational expressions thereof from the setting device 300. For example, the risk determination device 200 can calculate the relational expressions of the soil parameters by acquiring the soil parameters of each moisture amount in soil. Note that, as described in the second example embodiment, those relational expressions may be stored in the risk determination device 200 in advance.

FIG. 9 is a table showing an example of topographic data of the plurality of slopes (A to G). In this example, the slope A has a slope length of “5.6”, a sliding layer depth of “0.5”, and a slope angle of “37.0”. The topographic data are data obtained by measuring the actual field, and is stored in the risk determination device 200 in advance. Note that the topographic data required for calculating the safety factor may differ depending on the slope stability analytic expression used for calculation of the safety factor.

In the present example embodiment, the risk determination device 200 calculates the safety factor of the slope by use of the relational expressions and the topographic data described above. Herein, as an example, a method of calculating the safety factor by use of the modified Fellenius method is disclosed. A safety factor Fs obtained with the modified Fellenius method is expressed with Equation (1) in the following by use of the above-mentioned soil parameters (the soil clod weight W, the pore water pressure u, the viscosity c, and the internal friction coefficient φ) and a slope angle α of the slope. Note that the slope angle α may be a value set in advance.

$$\begin{matrix}[ Math . 1 ] & \\ Fs = \frac{c + ( W - u ) cos α tan φ}{W sin α} & ( 1 )\end{matrix}$$

Further, in the case of using vegetation data, the safety factor Fs may be calculated with Equation (2) in place of Equation (1), for example. In Equation (2), a viscosity cv indicates a component originated from a root system of vegetation among viscosities. Further, an upper load Wv indicates a load originated from the vegetation against the slope. Note that, when the safety factor is calculated, a specific method using the vegetation data is not limited to the example of Equation (2).

$$\begin{matrix}[ Math . 2 ] & \\ Fs = \frac{c + {c}_{v} + ( W + {W}_{v} - u ) cos α tan φ}{( W + {W}_{v} ) sin α} & ( 2 )\end{matrix}$$

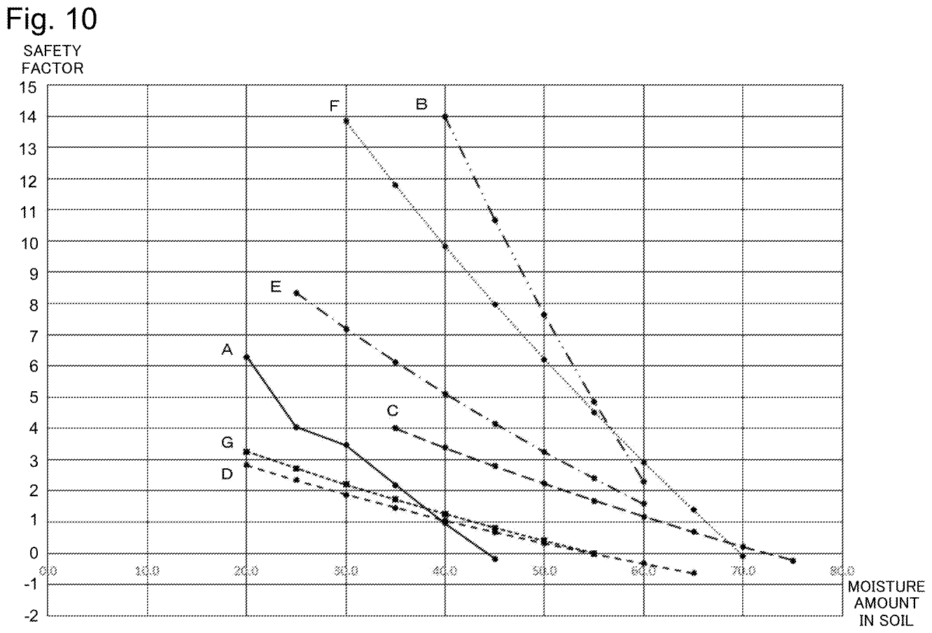

FIG. 10 is a graph illustrating relationships between the safety factors and the moisture amounts in soil which are calculated for the plurality of slopes (A to G). Note that, in this example, it is assumed that a threshold value of the safety factor, which is used for determining the collapsing risk, is “1.0”. In this case, the slopes B and E have the safety factors at the time of saturation, which are equal to or greater than the threshold value. Therefore, it can be said that the slopes B and E have a lower risk of slope collapsing as compared to the other exemplified slopes.

In view of such determination results, a user decides a slope to which a sensor is required to be installed (or an order of priority thereof). In the case of the example in FIG. 10, the slopes A, C, D, F, and G can be said as points where sensors are required to be installed preferentially to the slopes B and E. Further, when the slopes B and E are compared, it can be said that the slope having a higher safety factor at the time of saturation, i.e., the slope B has a lower risk of slope collapsing.

As described above, the risk determination system 30 according to the present example embodiment can exert the actions and effects similar to those in the first example embodiment and the second example embodiment. Further, according to the risk determination system 30, the data required for determination of the collapsing risk can be acquired through the adding water test.

For example, in the case where the safety factor is measured experimentally at the actual field in order to determine whether or not to set the slope as a monitor target, it is required that a sensor be installed and withdrawn. However, there may be a case where it is difficult to install a sensor on such a slope that slope collapsing may occur. Further, there may be a case where the change in moisture condition is required to depend on natural phenomena (rain and the like) when a transition of the safety factor is measured at the actual field. It can be said that the risk determination system 30 according to the present example embodiment is advantageous in terms of cost and safety as compared to the determination requiring such a measurement at the actual field.

### Modified Examples

For example, the first to third example embodiments described above can adopt the following modifications. Those modification examples can be combined appropriately as needed.

### Modified Example 1

The second calculation unit 120 may calculate another index indicating the safety of the slope in place of the safety factor. This index is an index similar to the safety factor or an index calculated based on the safety factor. For example, the second calculation unit 120 may be configured to calculate an index that is similar to and substitutable to the safety factor in place of the safety factor itself.

### Modified Example 2

A specific hardware configuration of the device according to the present disclosure may not be limited to a specific configuration. In the present disclosure, the constituent elements described functionally by use of the block diagrams may be achieved by various types of hardware and software, and are not necessarily related to specific configurations. Further, the constituent element described with one block in the present disclosure may be achieved by a plurality of collaborating pieces of hardware.

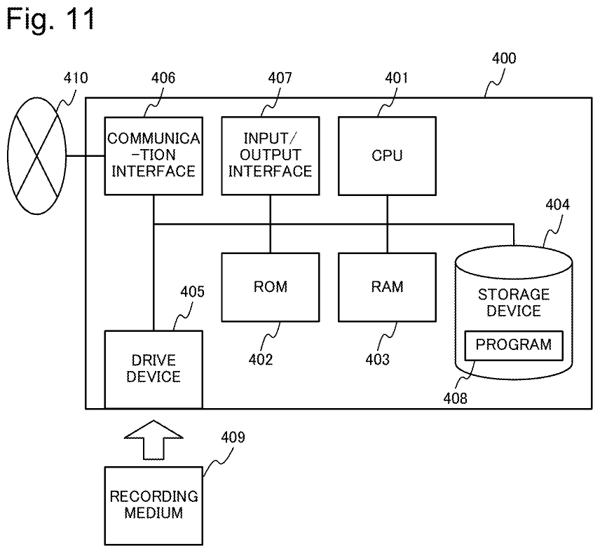

FIG. 11 is a block diagram illustrating one example of a hardware configuration of a computer device 400 achieving the device according to the present disclosure. The computer device 400 includes a central processing unit (CPU) 401, a read only memory (ROM) 402, a random access memory (RAM) 403, a storage device 404, a drive device 405, a communication interface 406, and an input/output interface 407.

The CPU 401 executes a program 408 by use of the RAM 403. The program 408 may be stored in the ROM 402. Further, the program 408 may be recorded in a recording medium 409 such as a memory card, may be read out by the drive device 405, or may be transmitted from an external device via a network 410. The communication interface 406 receives and transmits data with the external device via the network 410. The input/output interface 407 receives and transmits the data with peripherals (an input device, a display device, and the like). The communication interface 406 and the input/output interface 407 are capable of functioning as constituent elements for acquiring or outputting the data.

The device according to the present disclosure may be achieved by the configuration (or a part of the configuration) illustrated in FIG. 11. For example, the CPU 401 can achieve the function of calculating the parameters indicating the soil condition (the first calculation unit 110 and the like), the function of calculating the safety factor of the slope (the second calculation unit 120 and the like), and the function of determining the risk of slope collapsing (the determination unit 130 and the like) by using the RAM 403 as a temporary storage region to execute the program 408.

Note that the constituent elements in the present disclosure may be configured by a single circuitry (a processor and the like), or may be configured by a combination of a plurality of circuitries. The circuitry described herein may be either of dedicated one or a general-purpose one. For example, a part of the device according to the present disclosure may be achieved by a dedicated processor, and other parts thereof may be achieved by a general-purpose processor.

In the example embodiments described above, the configuration described as the single device may be provided in a distributed manner to a plurality of devices. For example, by use of, for example, a technology of cloud computing and the like, the risk determination device 100 may be achieved by a plurality of collaborating computer devices. Further, any of the first calculation unit 110, the second calculation unit 120, and the determination unit 130 of the risk determination device 100 may be included in another device.

While the invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims.

This application is based upon and claims the benefit of priority from Japanese patent application No. 2017-004595, filed on Jan. 13, 2017, the disclosure of which is incorporated herein in its entirety by reference.

### REFERENCE SIGNS LIST

100, 200 Risk determination device
110 First calculation unit
120 Second calculation unit
130 Determination unit
Risk determination system
300 Setting device
310 Adding water unit
320 Measurement unit
330 Determination unit
340 Output unit
400 Computer device

1. A risk determination device, comprising:
a first calculation unit configured to calculate a parameter indicating a condition of soil constituting a certain slope, based on a relationship between the condition of the soil and a moisture condition of the soil and virtual data of the moisture condition;
a second calculation unit configured to calculate a safety factor of the slope by use of the parameter being calculated; and
a determination unit configured to determine a collapsing risk of the slope, based on the moisture condition in which the safety factor being calculated is less than a threshold value, and the moisture condition of the soil based on the virtual data when the moisture is saturated.

2. The risk determination device according to claim 1, wherein
the first calculation unit specifies a relational expression indicating the relationship between the condition of the soil and the moisture condition of the soil, based on the virtual data, and
calculates the parameter by use of the relational expression being calculated.

3. The risk determination device according to claim 1, wherein
the second calculation unit calculates the safety factor of the slope by use of the parameter being calculated and data indicating topography or vegetation of the slope.

4. The risk determination device according to claim 1, wherein
the virtual data includes a test value or a literature value of a soil moisture amount of the soil.

5. The risk determination device according to claim 4, wherein
the virtual data include the test value of the soil moisture amount which is acquired by adding water to a sample of the soil until the soil moisture amount of the sample is saturated.

6. The risk determination device according to claim 1, wherein
the parameter includes at least any one of a soil clod weight, a pore water pressure, viscosity, and an internal friction coefficient of the soil.

7. (canceled)

8. A risk determination method, comprising:
calculating a parameter indicating a condition of soil constituting a certain slope, based on a relationship between the condition of the soil and a moisture condition of the soil and virtual data of the moisture condition;
calculating a safety factor of the slope by use of the parameter being calculated; and
determining a collapsing risk of the slope, based on the moisture condition in which the safety factor being calculated is less than a threshold value, and the moisture condition of the soil based on the virtual data when the moisture is saturated.

9. A non-transitory computer-readable recording medium which stores a program causing a computer to execute:
a step of calculating a parameter indicating a condition of soil constituting a certain slope, based on a relationship between the condition of the soil and a moisture condition of the soil and virtual data of the moisture condition;
a step of calculating a safety factor of the slope by use of the parameter being calculated; and
a step of determining a collapsing risk of the slope, based on the moisture condition in which the safety factor being calculated is less than a threshold value, and the moisture condition of the soil based on the virtual data when the moisture is saturated.
